(19) United States
(12) Patent Application Publication
(10) Pub. No.: US 2021/0148909 A1
(43) Pub. Date: May 20, 2021

(54) OPTIMIZATION OF AN ACTIVE PCSK9 ASSAY

(71) Applicant: NORTH CAROLINA CENTRAL UNIVERSITY, Durham, NC (US)

(72) Inventor: Dayami LOPEZ, Durham, NC (US)

(73) Assignee: NORTH CAROLINA CENTRAL UNIVERSITY, Mumbai, NC (US)

(21) Appl. No.: 16/616,712

(22) PCT Filed: May 31, 2017
(86) PCT No.: PCT/US2017/035216

§ 371 Date: Nov. 25, 2019

(51) Int. Cl. G01N 33/573

(52) U.S. Cl. CPC G01N 33/573; G01N 2333/96433

(57) ABSTRACT

An assay and a method for detecting how much active PCSK9 is available in a sample to bind to the LDL receptor. Active PCSK9 is PCSK9 that is not bound to a LDL receptor and is available to bind to a LDL receptor. An aspect of the assay and method involves the use of LDL receptor and a PCSK9 specific antibody to identify, detect or quantify the PCSK9/LDL receptor complexes.

### BACKGROUND OF THE INVENTION

Hypercholesterolemia (high levels of total and low density lipoprotein (LDL) cholesterol), the primary cause of atherosclerotic-related diseases, is still considered a severe health problem worldwide with, according to the CDC Report from 2014, more than 85 million patients in the United States. The major determinant of plasma LDL levels is the hepatic LDL receptor. The expression of this receptor is controlled by the proprotein convertase subtilisin/kexin-9 (PCSK9) (1, 2). PCSK9 is synthesized mainly by liver cells and secreted into the serum (2-4). Once in the serum, the PCSK9's C-terminal domain interacts with the LDL receptor's epidermal growth factor-like repeat A (EGF-A) domain at the surface of cells (2-4). After this interaction, the PCSK9/LDL receptor complex enters the endosomal pathway (3, 4). Unlike the binding of a lipoprotein particle to the LDL receptor, the PCSK9's affinity for the receptor is increased due to the acidic pH of the endosome (4). Failure to release PCSK9 in the endosome prevents receptor recycling directing the LDL receptor to be degraded within the lysosome (4).

Statins, the first line of treatment for hypercholesterolemic patients, have been successfully used to improve the survival of individuals at risk of developing atherosclerosis (5). Nevertheless, 16-53% of the patients fail to reach their goal in LDL cholesterol levels upon statin treatment (statin resistance). This is mostly due to an unwanted increase in PCSK9 protein levels (6). The rate of the LDL receptor protein degradation is also increased as a result of the upregulation of PCSK9 levels (7). In fact, in humans and animal models, it has been shown that statin treatment increases plasma PCSK9 levels resulting in a partial attenuation of the effects of statins on the LDL receptor expression (6, 8-12). Thus, PCSK9 appears to be responsible for statin resistance reported in some patients. Interestingly, PCSK9 has been identified as one of the genes in which polymorphisms are associated with resistance to statins (13).

These results, and the discovery of the connection between loss-of-function (LOF) mutations of PCSK9, hypocholesterolemia, and a decreased risk of cardiovascular disease (14), justified the development of PCSK9 inhibitors to treat hypercholesterolemia, especially in combination with statins. Currently, two therapeutics, Praluent (alirocumab) (https://www.praluent.com) and Repatha (evolocumab) (https://www.repatha.com) with the ultimate purpose of preventing the PCSK9-dependent degradation of the LDL receptor have been approved by the US FDA to treat hypercholesterolemic patients.

### SUMMARY OF THE INVENTION

To determine the efficiency of these therapeutics as well as other pharmaceuticals and treatments against PCSK9 and to also identify patients that may qualify for these and other treatment options, such as statins, an assay to determine active PCSK9 levels in samples is needed. An assay to determine total PCSK9 is described in Arch. Biochem. Biophys. 545:124-132.2014. The disclosure of this article is incorporated herein by reference in its entirety. An assay that detects total PCSK9 detects all of the PCSK9 present in a sample (active and inactive PCSK9). The assay described below is intended to be used to determine active PCSK9 levels in specimens. Among other uses, an assay to determine active PCSK9 levels can be used to confirm whether drugs for inhibiting PCSK9 work as expected, and/or to determine which patients should be treated with these drugs and/or other treatments.

### DETAILED DESCRIPTION OF THE FIGURES

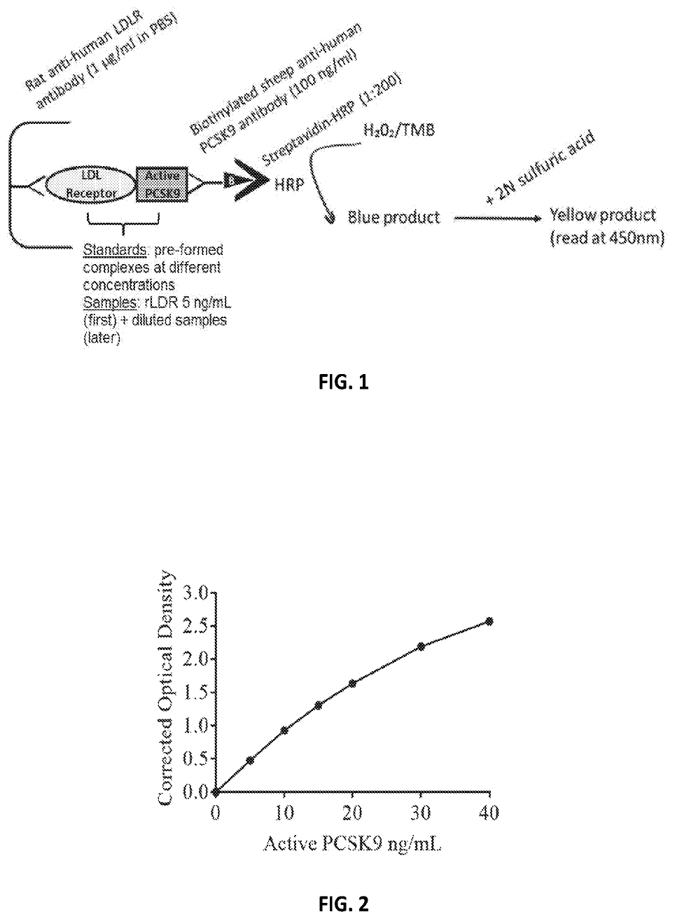

FIG. 1. shows a schematic of an active PCSK9 assay.

FIG. 2. shows the typical standard curve for an active PCSK9 assay.

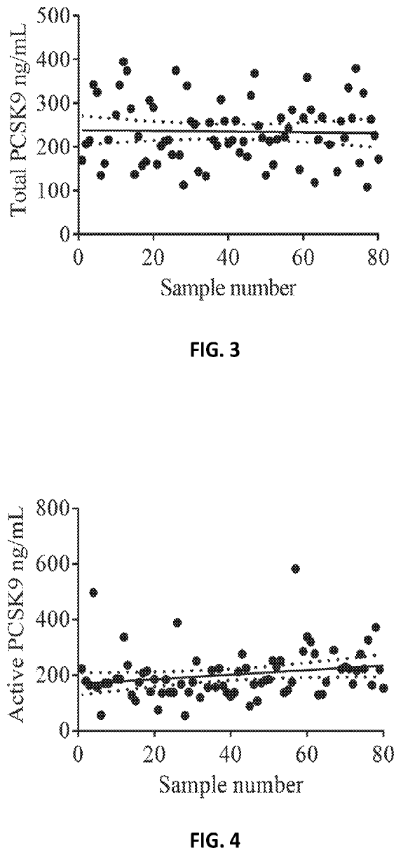

FIG. 3. shows total PCSK9 levels in 80 random human serum samples.

FIG. 4. shows active PCSK9 levels in 80 random human serum samples.

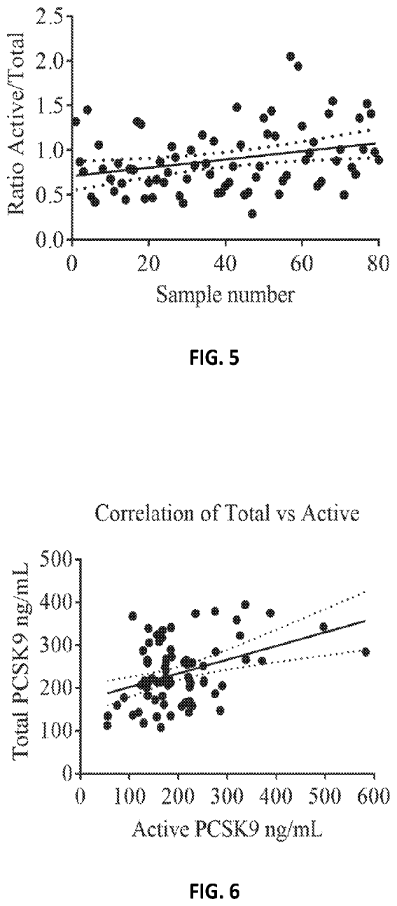

FIG. 5. shows the ratio of active PCSK9 to total PCSK9 in 80 random human serum samples.

FIG. 6. shows a correlation of total PCSK9 levels versus active PCSK9 levels in human serum samples.

### DETAILED DESCRIPTION OF THE INVENTION

Other than in any operating examples, or where otherwise indicated, all numbers expressing quantities of ingredients, reaction conditions, times, and so forth used in the specification and claims are to be understood as being modified in all instances by the term “about.” Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Accordingly, unless indicated to the contrary, the numerical parameters set forth in the specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

As used in this specification and the appended claims, the articles “a,” “an,” and “the” include plural referents unless expressly and unequivocally limited to one referent.

In order that the disclosure may be readily understood and put into practical effect, reference is made to exemplary embodiments as illustrated with reference to the accompanying figures. The figures together with a detailed description below, are incorporated in and form part of the specification, and serve to further illustrate the embodiments and explain various principles and advantages, in accordance with the present disclosure. The disclosure and various examples of the present invention as presented herein are each understood to be non-limiting with respect to the scope of the invention.

As used in the following description and claims, the following terms have the meanings indicated below.

The term antibody is used herein in the broadest sense and can cover monoclonal antibodies, polyclonal antibodies, multivalent antibodies, multispecific antibodies and antibody fragments so long as they exhibit the desired biological activity.

The words assay and test can be used interchangeably. Immunoassay is a type of assay.

Carrier is used in the broadest sense and can mean but is not limited to a well plate, a 96 well plate, a high affinity 96 well plate, an ELISA plate, a high binding ELISA plate, a microtiter plate or another similar plate or the like that can be used to perform the method described herein.

The terms detect, detecting and detection are used in the broadest sense to include qualitative and/or quantitative measurements. Detection can be conducted using a moiety or technique used to detect, for example, PCSK9 or PCSK9/LDL complexes.

Sample is used in the broadest sense and a sample is typically selected from blood, plasma, serum, cell culture medium, or a combination thereof.

An assay and a method were developed to detect how much active PCSK9 is available in a sample to bind to the LDL receptor. Active PCSK9 is PCSK9 that is not bound to a LDL receptor and is available to bind to a LDL receptor.

The method comprises contacting a sample which contains or is expected to contain PCSK9 for a time and under conditions sufficient for an antigen-antibody complex to form and then detecting the formation of the antigen-antibody complex. The conditions are selected to maximize the sensitivity and usefulness of the assay.

An aspect of this invention is an assay that is an indirect sandwich ELISA that involves the use of LDL receptor and a PCSK9 specific antibody to identify, detect or quantify the PCSK9/LDL receptor complexes. In an aspect of the invention, PCSK9/LDL receptor complexes are formed by adding a sample to a carrier or plate containing the LDL receptor.

The assay and method involve the use of an LDL receptor-specific antibody to bind the LDL receptors to a plate. The LDL receptor can be a recombinant LDL (rLDL) receptor. After active PCSK9 is bound to the LDL receptor, the PCSK9/LDL receptor complex is detected using a PCSK9-specific antibody, which is indirectly detected using a detection agent. The PCSK9-specific antibody can be a biotinylated PCSK9-specific antibody. A detection agent such as streptavidin-HRP (horseradish peroxidase) or avidin-HRP can be used.

Following the addition of the detection agent, a reagent detecting agent is added. To stop color development, a different reagent is added. The reagent detecting agent may include a chromogenic substrate. The chromogenic substrate can be tetramethylbenzidine (TMB) or 2,2′-Azinobis[3-ethylbenzothiazoline-6-sulfonic acid]-diammonium salt (ABTS) or another chromogenic substrate. H₂O₂/TMB may be used as the reagent detecting agent. Color development can be stopped by a different reagent. Sulfuric acid or hydrochloric acid can be used to stop the color development. The intensity of the resulting color is directly proportional to the number of complexes formed between active PCSK9 and the LDL receptor.

A schematic representation of an assay according to the invention is illustrated in FIG. 1. A carrier or a well plate such as a high affinity 96 well plate can be used in the assay or method. A carrier or well plate is coated for at least several hours, preferably, overnight at room temperature with a LDL receptor antibody, such as a rat anti-human LDL receptor antibody, diluted in a buffer. After overnight incubation, the coating solution is aspirated, and the wells are washed multiple times with a wash buffer.

Blocking is carried out with a blocking solution. After the blocking solution is added, the plate is incubated for 1 to 2 hours, preferably, 1.5 hours at room temperature. The wells are then washed. Wells to be used in the detection of active PCSK9 are incubated with LDL receptor diluted in blocking solution. In an aspect of the invention, 5 ng/ml of LDL receptor or 5 ng/ml rLDL (recombinant LDL) receptor can be used. After coating and blocking, complex standards (PCSK9/LDL receptor complexes prepared in vitro) and diluted samples such as patient blood or serum, or cell culture medium samples are added to wells. Wells to be used for complex standards are incubated with an equal volume of blocking solution. The plate is covered with for example, plastic wrap or plate sealer; the plate is incubated for 1 hour at room temperature, and washed with the wash buffer. Incubation is then carried out at 37° C. for 1 hour to allow a complex to form between the LDL receptor and PCSK9 in the samples. The wells are then washed with the wash buffer.

Detection of PCSK9/LDL receptor complexes bound to the coating antibody (LDL receptor antibody) is carried out using a PCSK9 antibody. A PCSK9 antibody diluted in blocking buffer is added and the plate is incubated for 1 hour at room temperature. The PCSK9 antibody may be biotinylated. Non-limiting examples of a PCSK9 antibody that can be used is sheep anti-human PCSK9 antibody. A non-limiting example of a PCSK9 antibody is biotinylated sheep anti-human PCSK9 antibody. In an aspect of the invention, the concentration of the PCSK9 antibody is 100 ng/mL.

After incubation, the plate is washed with the wash buffer and the detection agent diluted in blocking buffer is added. Non-limiting examples of detection agents are streptavidin-HRP and avidin-HRP.

The detection agent is diluted in blocking buffer (for example in a ratio of 1:200) and added to the plate. The plate is incubated for 30 minutes at room temperature. Following the incubation, the plate is washed with the wash buffer. Reagent detecting agent (substrate solution) is added to the wells and incubated in the dark for 30 minutes at room temperature. A reagent detecting agent that can be used contains 0.01% H₂O₂ and 0.2 g/L tetramethylbenzidine. The reaction is stopped by adding a stopping agent, such as 1 or 2N sulfuric acid or 0.5 m hydrochloric acid.

The same wash buffer should be used in all washing steps of the assay or method. In an aspect of the invention, each time the carrier, wells or plate is washed with a wash buffer, it is washed three times.

A buffer that can be used in the assays and methods described in the specification and claims is phosphate buffered saline (PBS). A wash buffer that can be used in the assays and methods described in the specification and claims is PBS/0.05% Tween 20. A blocking solution/buffer that can be used in the assays and methods described in the specification and claims is 1% bovine serum albumin (BSA) in PBS.

The number and concentrations of complex standards (PCSK9/LDL receptor complexes prepared in vitro) added is determined based on the assay. In an aspect of the invention, the standards are diluted in the blocking buffer. For example, 8 standards can be prepared, with concentrations ranging between 0 and 40 ng/mL. Diluted samples of, such as patient serum or cell culture medium samples, are then added to corresponding wells. In an aspect of the invention, the samples are diluted using only PBS.

The optical density of the resulting colored product is determined using a plate reader with the correct filters to read at 450 and 540 nm. A plate reader such as a BMG Labtech PHERAstar™ plate reader can be used. Correction for optical imperfections in the plate are carried out by reading the plate at 540 nm, and these readings were subtracted from the 450 nm readings. A typical standard curve for this assay is shown in FIG. 2. LDL-receptor/PCSK9 complex standards used in this assay had concentrations from 0-40 ng/mL. The assay was relatively linear through the entire range of concentrations. R²=0.9996. The equation for this graph is Y=0.0928X+0.0051. Y corresponds to the corrected OD. X corresponds to the sample concentration.

The sample used is selected from blood, plasma, serum, cell culture medium, or a combination thereof. In certain embodiments, the sample is a sample of a subject, the subject may be a human or non-human. The non-human is preferably a mammal. The subject may be a patient.

In another aspect, samples were analyzed using the newly developed active PCSK9 assay (described here) and a variation of the total PCSK9 assay previously described in Arch. Biochem. Biophys. 545:124-132, 2014. A total PCSK9 assay detects all the PCSK9 present in a sample (active and inactive) since in this assay, PCSK9 specific antibody is coated on the plate.

To detect total PCSK9 protein levels in samples, a sandwich type ELISA was used. A well plate was coated with a PCSK9 antibody in buffer at room temperature. After overnight incubation, the coating solution was aspirated, and the wells were washed with a wash buffer. Blocking was carried out with a blocking buffer for one hour at room temperature. Following which the plate was washed with wash buffer. PCSK9/LDL standards (as described above) and diluted samples containing PCSK9 were added to the plate, the plate was covered and incubated at room temperature. The contents of the wells were discarded, and the plate was washed with wash buffer. Detection of the PCSK9 protein bound to the coating antibody was carried out with a different PCSK9 antibody diluted in blocking buffer. After washing with wash buffer, a detection agent in blocking buffer is added to wells. The plate was covered and incubated at room temperature. The plate is washed with wash buffer. A reagent detecting agent was added. The plate was covered and incubated at room temperature. The reaction was stopped by adding stopping reagent to each well. The plate was read at 450 and 540 nm in a plate reader to determine the optical density.

The same wash buffer should be used in all washing steps of the assay or method. In an aspect of the invention, each time the carrier, wells or plate is washed with a wash buffer, it is washed three times.

In another aspect of the invention, total PCSK9 protein levels in samples, is detected using a sandwich type ELISA. A well plate is coated with a PCSK9 antibody. The PCSK9 antibody is diluted in buffer. An example of a suitable dilution is (2 g/ml). A rat anti-human PCSK9 antibody can be used. The plate is incubated overnight at room temperature. After overnight incubation, the coating solution is aspirated, and the wells are washed three times with washing buffer. Blocking is carried out with blocking buffer for 1.5 hr at room temperature. Following which the plate is washed three times with washing buffer. PCSK9 standards with concentrations ranging from 0 to 20 ng/ml and diluted samples containing PCSK9 are added to the plate, the plate is covered and incubated for one hr at room temperature. Contents of the wells are discarded, and the plate is washed three times with wash buffer. Detection of the PCSK9 protein bound to the coating antibody is carried out with a different PCSK9 antibody (PCSK9 detection antibody). The antibody may be biotinylated. Non-limiting examples of a PCSK9 antibody that can be used is sheep anti-human PCSK9 antibody. A non-limiting example of a PCSK9 antibody is biotinylated sheep anti-human PCSK9 antibody. The antibody is diluted in blocking buffer, for example at a concentration of (100 ng/ml). The plate is incubated for one hour at room temperature. After this, the plate is washed three times with washing buffer, and detection agent, for example streptavidin-HRP or avidin-HRP, in blocking buffer is added to wells. The plate was covered and incubated for 20 minutes at room temperature. The plate is washed three times with washing buffer followed by incubation with reagent detecting agent (substrate solution) containing chromogenic agent at room temperature. The reaction was stopped with stopping reagent added to each well. The plate was read at 450 and 540 nm in a plate reader to determine the optical density.

A buffer that can be used in this assay or method is phosphate buffered saline (PBS). A wash buffer that can be used in this assay or method is PBS/0.05% Tween 20. A blocking solution/buffer that can be used is 1% bovine serum albumin (BSA) in PBS.

Non-limiting examples of detection agents are streptavidin-HRP and avidin-HRP.

A non-limiting detecting agent that can be used contains 0.01% H₂O₂ and 0.2 g/L tetramethylbenzidine. A non-limiting example of a stopping agent is 1N or 2N sulfuric acid or 0.5M hydrochloric acid.

When the assay to detect total PCSK9 is to be used in comparison with the assay to detect active PCSK9, the same buffer, wash buffer, blocking buffer, detection agent, reagent detecting agent and stopping reagents should be used.

When the total PCSK9 assay and the active PCSK9 assays are used to test a sample, it is preferred that the same PCSK9 detection antibody is used in both assays. The use of the same PCSK9 antibody provides better comparative results.

In an embodiment of the invention, after the initial blocking is carried out with a blocking buffer and the plate is washed, the plate can be stored at a low temperature such as 4° C. for up to two weeks). There does not have to be liquid in the wells for storage.

The present disclosure further relates to a kit that can be used for detecting and optionally quantifying PCSK9 in a sample. The kit can include components selected from a capture antibody such as a LDL receptor antibody, a detection antibody such as a PCSK9 antibody, detecting agent, a reagent detecting agent, buffer, wash buffer, blocking buffer and stopping solution or any combination thereof. The kit can also include a plate or carrier. The kit can also include PCSK9 standards. The kit can include instructions for use.

It is thought that the clinical use of the active PCSK9 assay described in this application alone or in combination with other assays such as total PCSK9 assay described in Arch. Biochem. Biophys. 545:124-132, 2014 or the total PCSK9 assay described in this specification will be able to be used to identify patients that are more likely to develop cardiovascular diseases, intolerance or resistance to statins and susceptibility to diabetes, before starting a statin treatment. The availability of tests that could provide this type of information could be used by doctors to identify better treatment options for a patient, thereby, reducing the risk of side-effects. These tests could be used to monitor patients' progress after starting treatment.

These tests could be used to determine if a patient will benefit from approved PCSK9 inhibitors such as Praluent and Repatha. Even though these inhibitors seem to be more efficient than statins in reducing hypercholesterolemia in a variety of patients, their cost prevents many patients from having complete access. The assays that are described here could also identify which patients would benefit from these new drugs before, for example, they incur high financial costs or side-effects.

Another potential use of the combination of the total PCSK9 and active PCSK9 assays will be to identify patients with gain-of-function (a ratio greater than the mean) and loss-of-function (ratio lower than the mean) PCSK9 mutations. This could replace or supplement genetic tests. Differences in total and active PCSK9 levels due to phosphorylation, alternative splicing or furin-cleavage of PCSK9 could be determined using these tests. The following examples are intended to illustrate various aspects of the invention, and should not be construed as limiting the invention in any way.

### EXAMPLES

### Detailed Assay Description

### Active PCSK9 Assay

List of Supplies and how to Prepare them:

HiEh binding ELISA plates. Cat #2592, Corning (also available via Fisher Scientific) PBS (ph=7.2 to 7.4). (buffer) dissolve the indicated amounts of each chemical in the indicated volume of deionized water (final concentrations=137 mM NaCl, 2.7 mM KCl, 8.1 mM Na₂HPO₄, 1.5 mM KH₂PO₄). Check pH and if not between the indicated range, adjust. Filter the solution using a 0.2 μM filter.

| 1 liter | | 750 mL | | 500 mL | | 250 mL | |
|---|---|---|---|---|---|---|---|
| 8.006 | g NaCl | 6.0045 | g NaCl | 4.003 | g NaCl | 2.0015 | g NaCl |
| 0.201 | g KCl | 0.15 | g KCl | 0.1 | g KCl | 0.05 | g KCl |
| 1.15 | g Na₂HPO₄ | 0.8625 | g Na₂HPO₄ | 0.575 | g Na₂HPO₄ | 0.2875 | g Na₂HPO₄ |
| 0.204 | g KH₂PO₄ | 0.153 | g KH₂PO₄ | 0.102 | g KH₂PO₄ | 0.051 | g KH₂PO₄ |
| 1000 | mL DiH₂O | 750 | mL DiH₂O | 500 | mL DiH₂O | 250 | mL DiH₂O |

LDLR Coatinn antibody. Cat #MAB2148-500, R&D Systems or Fisher Scientific. Reconstitute the Capture (Coating) Antibody with PBS to a final concentration of 500 μg/mL. For example, for a 500 μg vial, add 1 mL of PBS for a final concentration of 500 μg/mL. DO NOT VORTEX. Microcentrifuge for 30 seconds to collect everything in the bottom of the tube. Split into 20 μl aliquots, Store at −80° C. A 20 μl of 500 μg/mL diluted into 10 mL of PBS (final concentration=1 g/mL) is enough to coat one plate.

Wash Buffer, prepare 500 mL of PBS supplemented with 0.05% of Tween 20. Store at room temperature.

BSA (EUSA rade) Cat #sc-2323, Santa Cruz Biotechnology, or Cat #MP219989890, Fisher Scientific. Prepare by dissolving 2 g of BSA in 100 mL of PBS.

Reanent Diluent (also blocking buffer): 1% BSA in PBS. Store at 4° C. Use within three days.

rLDLR CF, (recombinant LDL receptor protein Carrier Free) Cat #2148-LD, R&D Systems or Fisher Scientific. Dilute 25 μg vial with 1 mL of PBS for a final concentration of 25 μg/mL. DO NOT VORTEX. Mix by inverting and microcentrifuge for 30 seconds to collect everything in the bottom of the tube. Split into 100 μl aliquots, Store at −80° C. Then dilute to 2.5 μg/mL with PBS. To make this dilution, add 100 μL of rLDLR (25 μg/mL stock)+900 μl of PBS. DO NOT VORTEX. Mix by inverting and then split into 100 μl aliquots, Store at −80° C. For experiments, use rLDLR CF at 5 ng/mL, which is made by diluting 25 μL of the 2.5 μg/mL stock with 12.5 mL of reagent diluent.

Complex standard, (PCSK9/LDL receptor complexes prepared in vitro), Cat #842916 R&D Systems or Fisher Scientific. Dilute with reagent diluent for a final concentration of 510 ng/mL. For example, for a 135 ng vial add 265 μL of reagent diluent. DO NOT VORTEX. Mix by inverting and microcentrifuge for about 30 seconds to collect everything in the bottom of the tube. Divide into 140 μL aliquots. Store aliquots at −80° C. until ready to use them. Two 80 μL aliquots will be required to prepare a set of standards.

PCSK9 Detection antibody, Cat #BAF3888, R&D Systems or Fisher Scientific. Reconstitute the Detection Antibody with Reagent Diluent to a final concentration of 72 μg/mL. For example, for a 50 μg sample, add 714 μL of Reagent Diluent. Mix by inverting. After reconstitution, divide this antibody into 15 μl aliquots and save at −80° C. Each vial will be enough for a 96 well plate.

Streptavidin-HRP, Part #890803; R&D systems or Fisher Scientific. Within 30 minutes of use, prepare 10 mL of working dilution (per plate) of streptavidin-HRP in Reagent Diluent according to the instructions on the stock vial (usually 1:200 dilution). To prepare 10 mL of a 1:200 dilution, dilute 50 μL of the Streptavidin-HRP solution in 10 mL of reagent diluent.

Substrate solution (reagent detecting anent). (0.01% H₂O₂ and 0.2 g/L tetramethylbenzidine or TMB), Part #34021, Thermo Scientific Pierce. Prepare the Substrate solution within 5 minutes of adding it to the plates by mixing 5 mL of Color Reagent A (hydrogen peroxide) solution with 5 mL of Color Reagent B (TMB) per plate. Cover the tube with foil while waiting.

Stop solution: 2N H₂SO₄ or 0.5 M hydrochloric acid.

### Example 1

Procedure:

Day #1—Coatinn with LDLR specific antibody:

a) Prepare enough LDLR Capture antibody to coat one plate: dilute a 20 μL aliquot in 10 mL of PBS and mix by inverting at least six times. DO NOT VORTEX.
b) Add 100 μL of diluted Capture antibody (final concentration for coating=1.0 μg/mL) to each well in the ELISA plates. Cover the plate with plastic wrap and incubate overnight at room temperature with shaking.

Day #2—Blocking:
a) On the next day, remove the plate from the overnight incubation. Aspirate each well and wash with Wash buffer for a total of three times. Note: To wash, add 300 μL wash buffer to all the wells and then aspirate for a total of three times.
b) Add 150 μL of Reagent Diluent (blocking buffer or blocking solution) (PBS/1% BSA) to each well. Cover the plate with plastic wrap and incubate at room temperature with shaking for 1.5 hours.
c) Repeat the washing step three times.
d) At this point, the plate could be either stored at 4° C. covered with plastic wrap (use within two weeks) or utilized in the next step. Do not need to have liquid in the wells for storage.

The addition of rLDLR to detect active PCSK9:
a) Add 100 μL of Reagent diluent to the wells that will be used for standards only. To the wells that will be used to detect active PCSK9 in samples, add 100 μL of the rLDLR (5 n/mL solution prepared as described above).
b) Cover the plate with plastic wrap and incubate 1 hour at room temperature.
c) Wash three times as described above.

Sample preparation: Standards and samples can be prepared while waiting for the blocking step described above. Prepare all the samples at room temperature.

### 1. Prepare Standards as Follow:

| Std | Reagent Diluent | Standard | | Concentration |
|---|---|---|---|---|
| Number | (μL) | (μl) | | (pg/mL) |
| 1 | 921.6 | 78.4 | of the 510 stock | 40,000 |
| 2 | 941 | 59 | of the 510 stock | 30,000 |
| 3 | 500 | 500 | of std 1 | 20,000 |
| 4 | 500 | 500 | of std 2 | 15,000 |
| 5 | 500 | 500 | of std 3 | 10,000 |
| 6 | 500 | 500 | of std 5 | 5,000 |
| 7 | 500 | 500 | of std 6 | 2,500 |
| 8 | 1000 | 0 | | 0 (also |
| | | | | called blank) |

Mix well each sample (by inverting several times) before taking the amount that is needed to prepare the next standard. Previous preparation of the LDL receptor/PCSK9 complex ensures that each molecule of PCSK9 that is detected is bound to the LDL receptor.

Prepare samples as follow: Remove samples from −80° C. and let them melt at room temperature. Dilute them as follow:

Human serum samples: dilute 1:100 (10 μl of sample—mix well by inverting several times before taking the 10 μl aliquot—in 990 ul of PBS). Mix the samples several times by inverting before adding them to the plate. Save any remaining original protein sample at −80° C. More dilutions will be performed if necessary.

Medium samples—96 well plates: dilute 1:4 (50 μL of sample into 150 μL of PBS). More dilutions will be carried out if necessary.

Medium samples—6 or 12 well plates: dilute 1:10 (100 μL of the sample in 900 μL of PBS).

More dilutions will be carried out if necessary.

Medium samples —flasks: dilute 1:50 (20 μL of the sample in 980 μL of PBS).
Assay: All the samples and solutions need to be at room temperature before being added to the plates.
1. Remove unnecessary strips from the plate(s) and store them at 4° C. covered with wrap. Prepare a template of the assay.
2. To the remaining strips, add 100 μL of standards and samples in duplicate, side-by-side, according to the template that was prepared. For some experiments, samples will be run in singlets or triplicates, so it is important to establish a template of the samples. Here is an example of a template for an experiment with 16 samples:

| | 1 | 2 | 3 | 4 | 5 | 6 | 7 | 8 | 9 | 10 | 11 | 12 |
|---|---|---|---|---|---|---|---|---|---|---|---|---|
| A | Std 1 | Std 1 | Sample1 | Sample1 | Sample9 | Sample9 | | | | | | |
| B | Std 2 | Std 2 | Sample2 | Sample2 | Sample10 | Sample10 | | | | | | |
| C | Std 3 | Std 3 | Sample3 | Sample3 | Sample11 | Sample11 | | | | | | |
| D | Std 4 | Std 4 | Sample4 | Sample4 | Sample12 | Sample12 | | | | | | |
| E | Std 5 | Std 5 | Sample5 | Sample5 | Sample13 | Sample13 | | | | | | |
| F | Std 6 | Std 6 | Sample6 | Sample6 | Sample14 | Sample14 | | | | | | |
| G | Std 7 | Std 7 | Sample7 | Sample7 | Sample15 | Sample15 | | | | | | |
| H | Std 8 | Std 8 | Sample8 | Sample8 | Sample16 | Sample16 | | | | | | |

3. Cover the plate with plastic wrap and incubate at 37° C. with shaking for 1 hour.
4. Wash plates three times as indicated above.
5. Dilute a 15 μL aliquot of Detection antibody in 10 mL of Reagent Diluent and mix by inverting at least six times. Add 100 μL of diluted Detection antibody (final concentration in the plate=100 ng/mL) to each well. Cover the plate with plastic wrap and incubate 1 hour at room temperature.
6. Wash plates three times with washing buffer.
7. Add 100 μL of the diluted streptavidin-HRP (see instructions above) to each well. Cover the plate with plastic wrap and incubate 30 minutes at room temperature. Cover plate with aluminum foil or aluminum sealing tape to prevent direct exposure to light.
8. After the incubation, wash three times with washing buffer.
9. Add 100 μL of the Substrate solution (reagent detecting agent) (see above instructions) to each well. Cover the plate with plastic wrap and aluminum foil and incubate 30 minutes at room temperature. A blue color is seen at the end of this incubation.
10. After the incubation, add 50 μL of Stop solution (2N sulfuric acid) to each well. Gently tap the plate to ensure thorough mixing. A yellow color should be seen.
11. Read the plate at 450 and 540 nm.
12. Calculations:
   Per sample, subtract the 540 nm readings from the 450 nm readings (corrected readings).
   Average the duplicate corrected readings for each sample.
   Per sample, subtract the averaged corrected blank (from standard curve; 0 ng/mL) from all the averaged corrected OD readings (final ODs). After this correction, the final OD for the standard “0” should be zero.
   Plot the standard final ODs (y-axis) versus the concentration (x-axis) using scattered plot.

| Std | Final corrected ODs | Concentration |
|---|---|---|
| Number | (y-axis) | (ng/mL - x-axis) |
| 1 | | 40 |
| 2 | | 30 |
| 3 | | 20 |
| 4 | | 15 |
| 5 | | 10 |
| 6 | | 5 |
| 7 | | 2.5 |
| 8 | 0 | 0 |

Select the region of the graph that is linear and add a trendline (select “Display equation” and “Display R-squared value on chart” on the format trendline section).
The equation will have the following format:

y(OD)=slope*x(concentration)+intercept.
Calculate samples as follow:

Concentration=dilution factor*(final OD−intercept)/slope.

### Example 2

To detect total PCSK9 protein levels in medium samples, a sandwich ELISA was optimized using the Human PCSK9 DuoSet ELISA Development System from R&D Systems. A high-affinity 96 well plate was coated overnight with rat anti-human PCSK9 antibody (2 μg/ml) in PBS at room temperature. After overnight incubation, the coating solution was aspirated, and the wells were washed three times with PBS/0.05% Tween 20. Blocking was carried out with 1% BSA/PBS for 1.5 hr at room temperature. Washing with PBS/0.05% Tween 20 was done three times. One hundred μL of 8 rPCSK9 standards (concentration ranging from 0 to 20 ng/ml) and diluted medium samples (saved from treated wells above) containing PCSK9 were added to the plate, covered with a plastic wrap and incubated with shaking for one hr at room temperature. Contents were discarded, and the plate was washed three times with PBS/0.05% Tween 20. Detection of the PCSK9 protein bound to the coating antibody was carried out with a biotinylated sheep anti-human PCSK9 antibody (100 ng/ml) diluted in blocking buffer for one hr at room temperature with gentle rotation. After washing three times with PBS/0.05% Tween 20, 100 μl of streptavidin-HRP diluted in blocking buffer (1:200) was added to wells. The plate was covered with aluminum foil and incubated for 20 minutes at room temperature with gentle rotation. Washing three times was done followed by incubation 15 minutes with 100 μl of the ELISA TMB substrate at room temperature with gentle rotation and the plate covered with aluminum foil. The reaction was stopped with 50 μl of 2 M sulfuric acid to each well. The plate was read at 450 and 540 nm in a plate reader to determine the optical density.

FIG. 3 shows the total PCSK9 levels in the random human serum samples. The total PCSK9 assay detected PCSK9 levels with a median of 220 ng/mL and a mean of 235 ng/mL. The range of total PCSK9 levels was 108-395 ng/mL.

The coefficient of variation between these human samples was 30.76%. The coefficient of variation between replicates for the same sample was 5.78%. Analysis and graphing were conducted using the GrapPad Prism 7 Software.

FIG. 4 illustrates the active PCSK9 levels in the same human samples analyzed using the active PCSK9 assay. The active PCSK9 assay detected PCSK9 levels with a median of 179 ng/mL and a mean of 202 ng/mL The range of active PCSK9 levels was 55-583 ng/ml. The coefficient of variation between these human samples was 44.13%. The coefficient of variation between replicates for the same sample was 2.388%. Analysis and graphing were conducted using the GrapPad Prism 7 Software.

FIG. 5 shows the ratios of active PCSK9 to total PCSK9 per sample. The values included in FIGS. 3 and 4 were used in these calculations. The ratio was calculated by dividing the value of active PCSK9 by the value of total PCSK9 for each sample. The range of active/total PCSK9 ratios was 0.29-2.05. Analysis and graphing were conducted using the GrapPad Prism 7 Software.

The ratio median was 0.82, whereas the mean was 0.90. This indicates that the majority of the PCSK9 protein (about 90%) present in the human serum samples was active. The coefficient of variation between the different ratios was 40.79%. The correlation between total and active PCSK9 levels in this population of samples was slightly positive (FIG. 6) with an R²=0.1556.

### REFERENCES

1. J. Davignon et al., The influence of PCSK9 polymorphisms on serum low-density lipoprotein cholesterol and risk of atherosclerosis. Curr. Atheroscler. Rep. 12:308-315. 2010.
2. M. C. McNutt et al., Antagonism of secreted PCSK9 increases low density lipoprotein receptor expression in HepG2 cells. J. Biol. Chem. 284:10561-10570, 2009.
3. Y. W. Qian et al., Secreted PCSK9 downregulates low density lipoprotein receptor through receptor-mediated endocytosis. J. Lipid Res. 48:1488-1498, 2007.
4. K. Tveten et al., Interaction between the ligand-binding domain of the LDL receptor and the C-terminal domain of PCSK9 is required for PCSK9 to remain bound to the LDL receptor during endosomal acidification. Hum. Mol. Genet. 21:1402-1409, 2012.
5. D. I. Chasman et al., Genetic determinants of statin-induced low-density lipoprotein cholesterol reduction: the Justification for the Use of Statins in Prevention: an Intervention Trial Evaluating Rosuvastatin (JUPITER) trial. Circ. Cardiovasc. Genet. 5:257-264, 2012.
6. H. E. Careskey et al., Atorvastatin increases human serum levels of proprotein convertase subtilisin/kexin type 9. J. Lipid Res. 49:394-398, 2008.
7. G. C. Ness et al., G. C., Z. Zhao, and D. Lopez. 1996. Inhibitors of cholesterol biosynthesis increase hepatic low-density lipoprotein receptor protein degradation. Arch. Biochem.

Biophys. 325: 242-248, 1996.
8. S. Rashid et al., Decreased plasma cholesterol and hypersensitivity to statins in mice lacking Pcsk9. PNAS USA 102:5374-5379, 2005.
9. G. Welder et al., High-dose atorvastatin causes a rapid sustained increase in human serum PCSK9 and disrupts its correlation with LDL cholesterol. J. Lipid Res. 51:2714-2721, 2010.
10. G. Dubuc et al., A new method for measurement of total plasma PCSK9: clinical applications. J. Lipid Res. 51:140-149, 2010.
11. Z. Awan et al., Rosuvastatin, proprotein convertase subtilisin/kexin type 9 concentrations, and LDL cholesterol response: the JUPITER trial. Clin. Chem. 58:183-189, 2012.
12. A. V. Khera et al., Effects of niacin, statin, and fenofibrate on circulating proprotein convertase subtilisin/kexin type 9 levels in patients with dyslipidemia. Am. J. Card. 115:178-182, 2015.
13. Z. Reiner, Resistance and intolerance to statins. Nutr. Metab. Cardiovasc. Dis., 24:1057-66, 2014.
14. J. Mayne et al., Novel loss-of-function PCSK9 variant is associated with low plasma LDL cholesterol in a French-Canadian family and with impaired processing and secretion in cell culture. Clin Chem. 57:1415-1423, 2011.

1. A method for detecting active PCSK9 in a sample, said method comprising: (a) adding a sample containing PCSK9 to a carrier coated with a LDL receptor-specific antibody and LDL receptor, (b) adding a PCSK9 antibody to detect active PCSK9 in the sample by detecting PCSK9/LDL receptor complexes.

2. A method for detecting active PCSK9 comprising coating a plate with a LDL receptor-specific antibody, adding LDL receptor, adding a sample containing PCSK9, adding PCSK9 antibody, and detecting PCSK9/LDL receptor protein complexes.

3. The method of claim 1, wherein the sample is selected from blood, plasma, serum, cell culture medium or a combination thereof.

4. The method according to claim 1, wherein the PCSK-9/LDL receptor complex is detected using a biotinylated PCSK9-specific antibody.

5. The method according to claim 1, wherein active PCSK9 is detecting using a detection agent selected from streptavidin-HRP or avidin HRP.

6. The method according to claim 1, wherein a reagent detecting agent comprises a chromogenic substance.

7. The method according to claim 6, wherein the chromogenic substance is ABTS (2,2′-Azinobis[3-ethylbenzothiazoline-6-sulfonic acid]-diammonium salt) or TMB (3,3′,5,5′-tetramethylbenzidine).

8. The method according to claim 1, wherein the reagent detecting agent is H₂O₂/TMB.

9. The method for detecting active PCSK9 in a sample as claimed in claim 1, further comprising: (a) adding a sample containing PCSK9 to a carrier coated with a LDL receptor-specific antibody and LDL receptor, (b) adding a PCSK9 antibody, (c) adding a detection agent, (d) adding a reagent detecting agent and (e) detecting active PCSK9 in the sample by detecting PCSK9/LDL receptor complexes.

10. The method according to claim 1, comprising contacting a biological sample derived from the subject with the PCSK9 for a time and under conditions sufficient for an antigen-antibody complex to form and then detecting the formation of an antigen-antibody complex.

11. A method for comparing total PCSK9 with active PCSK9 comprising determining the active PCSK9 using a method according to claim 1, and determining the amount of total PCSK9.

12. An assay for detecting active PCSK9 in a sample.

13. The assay according to claim 12, further comprising detecting active PCSK9 and total PCSK9.

14. A kit for detecting and optionally quantifying active PCSK9 in a sample comprising a detection antibody comprising a PCSK9 antibody and one or more of a capture antibody, a detection agent, a reagent detecting agent, buffer, wash buffer, blocking buffer and stopping solution.

15. The kit according to claim 14, further comprising one or more of a plate, carrier and instructions for use.

16. The kit according to claim 13, further comprising a second PCSK9 antibody for detecting total PCSK9.
